(19) United States
(12) Patent Application Publication
(10) Pub. No.: US 2006/0269621 A1
(43) Pub. Date: Nov. 30, 2006

(54) Novel therapeutic extracts and molecules for degenerative conditions

(75) Inventor: Villo Morawala Patell, Bangalore (IN)

Correspondence Address: HAMRE, SCHUMANN, MUELLER & LARSON, P.C., P.O. BOX 2902, MINNEAPOLIS, MN, 55402-0902, US

(73) Assignee: Avestha Gengraine Technologies Pvt.Ltd., Bangalore (IN)

(21) Appl. No.: 10/584,228

(22) PCT Filed: Jul. 23, 2004
(86) PCT No.: PCT/IB04/02531

§ 371 Date: Jun. 23, 2006

(30) Foreign Application Priority Data
Aug. 18, 2003 (WO) PCT/IB03/03861

(51) Int. Cl. A61K 36/18

(52) U.S. Cl. 424/725

(57) ABSTRACT

In this invention we have characterized plant extracts derived from common plants used in Indian traditional medicine using a) chemical fingerprinting by HPLC and b) etiology based in-vitro cell assays in order to reduce the extracts to minimum constituent to use for therapeutic purpose against degenerative conditions like cardiovascular diseases, Diabetes mellitus, Hypertension, All types of Cancers, Obesity & weight problems, Arthritis & pain, Alzheimer's disease, Multiple sclerosis & Autoimmune diseases, Asthma & Allergies, Osteoporosis & bone health, Parkinson's disease, Stress, Disorders & diseases of the eye. The current status of the extracts is that all of them have been chemically fingerprinted and have gone through assays to screen anti-diabetic activity. Thus currently we are applying for the specific leads that have shown very specific anti-diabetic activity. However we are also working in parallel, to test these extracts in other assays, for generating information of there applicability in the prevention or treatment of degenerative conditions as mentioned above. As and when we will have data to support disease specific application of individual extracts we will amend our claims accordingly. The present invention relates to a composition for curing or treating degenerative disorders, a process for the preparation of the same, and usage of the same and, more particularly, to the composition having as a major component a single extract or multiple extracts containing therapeutically relevant bioactive, obtained from a certain kind of plant/plants and being capable of lowering or reducing a level of glucose in the blood of a patient suffering from diabetes mellitus by alleviating or ameliorating various symptoms caused by or associated with the diabetes mellitus as well as preventing the glucose level in the blood of the diabetic patient with high certainty from arising again. The invention also relates to the in-vitro screening of plant extracts for anti-diabetic activities in multiple cell based and biochemical systems. Studies have shown eleven potential leads, which are very valuable as they could lead to bioactives having therapeutic activity in the treatment of like cardiovascular diseases, Diabetes mellitus, Hypertension, All types of Cancers, Obesity & weight problems, Arthritis & pain, Alzheimer's disease, Multiple sclerosis & Autoimmune diseases, Asthma & Allergies, Osteoporosis & bone health, Parkinson's disease, Stress, Disorders & diseases of the eye.

### FIELD OF THE INVENTION

The present invention relates to novel therapeutic plant extracts and molecules for the treatment of degenerative diseases like cardiovascular diseases, Diabetes mellitus, Hypertension, All types of Cancers, Obesity & weight problems, Arthritis & pain, Alzheimer's disease, Multiple sclerosis & Autoimmune diseases, Asthma & Allergies, Osteoporosis & bone health, Parkinson's disease, Stress, Disorders & diseases of the eye and having antioxidant action.

According to the present invention plant extracts derived from common plants have been characterized using a) chemical fingerprinting by HPLC and b) etiology based in-vitro cell assays in order to reduce the extracts to minimum constituent to use for therapeutic purpose against degenerative diseases like cardiovascular diseases, Diabetes mellitus, Hypertension, All types of Cancers, Obesity & weight problems, Arthritis & pain, Alzheimer's disease, Multiple sclerosis & Autoimmune diseases, Asthma & Allergies, Osteoporosis & bone health, Parkinson's disease, Stress, Disorders & diseases of the eye and having antioxidant action. The extracts have been chemically fingerprinted and have gone through assays to screen anti-diabetic activity. Specific leads that have shown very specific anti-diabetic activity.

However the present invention also includes testing these extracts in other assays, for generating information of there applicability in the prevention or treatment of degenerative conditions as mentioned above.

### BACKGROUND OF THE INVENTION

Degenerative diseases develop over a period of time, with the symptoms and signs becoming progressively worse and the affected person's life becoming increasingly and generally irreversibly affected.

Although any part of the body can be involved, three systems in particular are prone to debilitating degenerative diseases—the nervous system, the muscular system, and the skeletal system.

The classification of degenerative disorders of the nervous system is difficult and somewhat arbitrary. Many of these conditions are familial or hereditary, breed-related, and involve degeneration of the nervous system within the first few months after birth. Premature degeneration of any component of the CNS, such as neurons, myelin sheaths or axons, can be considered under the broad panoply of abiotrophies which are disorders associated with an inherent lack of vital trophic or nutritive factor(s).

Disorders of muscle function include weakness, cramps (painful spasms), and degenerative changes amongst other problems.

Diabetes is often defined as a state in which homeostasis of carbohydrate and lipid metabolism is improperly regulated by insulin, which leads to abnormalities in the assimilation of carbohydrates in the body. This results primarily in elevated fasting and post-prandial blood glucose levels. If this imbalanced homeostasis does not return to normalcy and continues for a protracted period of time, it leads to a condition known as hyperglycemia that will in due course turn into a syndrome called Diabetes mellitus.

Diabetes mellitus can be defined as a group of syndromes characterized by hyperglycemia, altered metabolism of lipids, carbohydrates and proteins along with increasing risks of complication from vascular diseases. Very often, the seriousness of the disease is realized on the development of secondary symptoms that manifest in many forms, namely, difficulty in healing of wounds, neuropathy and so on. It is estimated currently, that over 143 million people all over the world suffer from Diabetes mellitus and in the case of most people, suffering from Diabetes; it is not properly diagnosed until irreversible complications set in. The long terms complications arising out of untreated or ineffectively treated Diabetes include, cardiovascular diseases and strokes, retinopathy, nephropathy and peripheral neuropathy.

The World Health Organization has projected India, as the country with the fastest growing population of diabetics and it is estimated that between 1995-2025, Diabetes patients in India will increase by 195%.

In order to maintain the blood glucose level within the normal range, diabetes mellitus is frequently treated using a combination of diet therapy with ergotherapy, insulin therapy, pharmaceutical therapy and so on. The major pharmacological agents available to lower blood glucose are—insulin seceretagogues and sensitizors.

Insulin Sensitizers are molecules that facilitate glucose uptake in insulin resistant conditions whereas the secretagogues increases the total amount of insulin secretion from the beta-cells of the pancreas. Hypoglycemic agents such as sulfonylureas and biguanides (metformin) are well known examples of secretagogues and sensitizors respectively (Aguilar-Bryan L, et al., Cloning of the beta cell high-affinity sulfonylurea receptor: a regulator of insulin secretion. Science 268:423-426, 1995; Lubbos H, et al., oral hypoglycemic agents in type II diabetes mellitus. American Family Physician. 52:2075-2078, 1995). The side effects of sulfonylureas include hypoglycemia, renal and hepatic disease, gastrointestinal disturbances, increased cardiovascular mortality, dermatological reactions, dizziness, drowsiness and headache. Biguanides lower blood glucose levels by reducing intestinal glucose absorption and hepatic glucose, but not by stimulating insulin secretion. The major side effects of biguanidine are lactic acidosis and increased cardiovascular mortality.

The other group of molecules that has immense potential are insulin mimetics and alpha-glucosidase inhibitors. The efficiency of the insulin mimetics are defined by the mimicking potential of unknown extracts where the activity is measured as the glucose uptake in differentiated 3T3 cells exposed to the test material only in absence of insulin. Alpha glucosidase inhibitors inhibit intestinal alpha glucosidases and consequently delay the digestion of sucrose and complex carbohydrates. The side effects of alpha glucosidase inhibitors include gastrointestinal side effects and hypoglycemia.

### OBJECTS AND SUMMARY OF THE INVENTION

The object of the present invention is to develop a multi-modal therapeutic approach for all degenerative diseases degenerative diseases like cardiovascular diseases, Diabetes mellitus, Hypertension, All types of Cancers, Obesity & weight problems, Arthritis & pain, Alzheimer's disease, Multiple sclerosis & Autoimmune diseases, Asthma & Allergies, Osteoporosis & bone health, Parkinson's disease, Stress, Disorders & diseases of the eye.

Future therapeutic strategies require the combination of various types of multiple agents. There is an urgent need to identify indigenous natural resources, procure them and study them in detail and their potential on newly identified targets in order to develop them as new therapeutics. Phytochemicals identified from traditional medicinal plants present an exciting opportunity to develop new kinds of therapeutics. Thus there is a need for a rationally designed interdisciplinary research programme, which could lead to the development of indigenous, renewable medicinal plant sources as practical and cost effective alternatives.

Medicinal preparations from herbal plants contain a variety of herbal and non-herbal ingredients that are believed to act on a variety of targets through various modes and mechanisms. The beneficial multiple activities like manipulating carbohydrate metabolism by various mechanisms, preventing and restoring integrity and function of beta cells, insulin releasing activity, improvement of the glucose-uptake, utilisation and the anti oxidant properties present in the medicinal plants, which offers exciting opportunities, to develop them into novel therapeutics. The theories of poly herbal formulation have the synergistic, potentiative, agnostic/antagonistic pharmacological agents within themselves due to the incorporation of plant medicines with diverse pharmacological actions. These pharmacological principles are known to work in a dynamic way to produce maximum therapeutic efficacy with minimum side effects. Traditional medicines ought not to be treated as a collection of therapeutic recipes. They are formulated and prepared, keeping in mind, the disease/sickness and the healing properties of the individual ingredients.

According to the present invention, etiology based cell assays and biochemical systems for the screening of plant extracts for anti-diabetic activities and degenerative disorders has been developed by developing formulations based on single or multiple extracts where the active constituents have been minimised to the least number that is required to retain activity.

The extracts and formulations prepared there-from addresses specific etiologies with scientifically validated leads for toxicity or efficacy from plants for the treatment of all degenerative disorders like cardiovascular diseases, Diabetes mellitus, Hypertension, All types of Cancers, Obesity & weight problems, Arthritis & pain, Alzheimer's disease, Multiple sclerosis & Autoimmune diseases, Asthma & Allergies, Osteoporosis & bone health, Parkinson's disease, Stress, Disorders & diseases of the eye.

The present invention is not to be limited in scope by the specific illustrations to the manifestations of diabetes but also to other degenerative disorders like cardiovascular diseases, Diabetes mellitus, Hypertension, All types of Cancers, Obesity & weight problems, Arthritis & pain, Alzheimer's disease, Multiple sclerosis & Autoimmune diseases, Asthma & Allergies, Osteoporosis & bone health, Parkinson's disease, Stress, Disorders & diseases of the eye.

Those skilled in the art will know, or be able to ascertain using no more than routine experimentation, many equivalents to the specific embodiments of the invention described herein. These and all other equivalents are intended to be encompassed by the following claims.

The assays done include—i) 3H deoxyglucose uptake by 3T3-L1, ii) MTT assay and iii) Alpha-glucosidase assay.

i) The de-oxyglucose uptake assay by fat cells or adipoctes (3T3-L1) help to identify the bioactives that helps to increase the glucose uptake by the fat cells. These extracts will help the diabetic individuals by quick removal of glucose from blood stream and simultaneously will help the fat cells to build a better storage of energy.

ii) The MTT assay is a measure of cellular respiratory physiology. It can estimate the cell proliferation rate and conversely, when metabolic events lead to apoptosis or necrosis, the reduction in cell viability.

iii) Alpha glucosidase inhibitors inhibit intestinal alpha glucosidases and consequently delay the digestion of sucrose and complex carbohydrates. The presence of molecules of this type in the bio-actives will ensure a safe uptake of sucrose added products to the diabetics by delaying the intestinal glucose uptake.

As of now, 11 leads from 10 different plants using the cell based assays to have functions of insulin mimetics, insulin sensitisors, cellular respiration boosters and alpha-glucosidase inhibitors. All these assays were screened for further in-vitro assays e.g. glucose metabolism by hepatocytes, glucose uptake by myocytes and insulin secretion from the pancreatic cells.

The present invention is not to be limited in scope by the specific embodiments and examples, which are intended as illustrations of a number of aspects of the invention and any embodiments which are functionally equivalent are within the scope of this invention. Those skilled in the art will know, or be able to ascertain using no more than routine experimentation, many equivalents to the specific embodiments of the invention described herein. These and all other equivalents are intended to be encompassed by the claims.

### BRIEF DESCRIPTION OF THE TABLES AND FIGURES

FIG. 1 illustrates the HPLC profile of 20% ethanol extract of Eugenia jambolana at various wavelengths to explain the process of complete metabolite profiling.

This process has been followed for all the extracts that we are discussing here. The actual measurements are done at 50 nm apart, here for easier representation we have only included data recorded at 200, 300, 400, 500, 600 and 700 nm.

FIG. 2 illustrates HPLC profile of the insulin mimetic at 270 nm as expressed in Table 8.

FIG. 3 illustrates the data from Table 8 expressed as a percent of the counts observed in the cells+insulin control.

FIG. 4: HPLC profile of insulin sensitizers at 270 nm as expressed in Table 9.

FIG. 5: The data from Table 9 are expressed as a percent of the counts observed in the cells+insulin control

FIG. 6: HPLC profile of four medicinal plant fractions that showed significant α-glucosidase inhibitory activities in the screening procedure.

FIG. 7: Shows that four medicinal plant fractions showed significant α-glucosidase inhibitory activities in the screening procedure.

Table 1: List of plants and their codes

Table 2: Table of Solvents used in the experiments and their codes

Table 3: Table of Plant part/tissue used in the experiment and their codes

Table 4: List of fractions of the plant extracts subjected to cell assays and the Counts per minute observed in study of insulin mimetic activity (without insulin) and study of insulin sensitizing activity (with insulin)

Table 5: Table showing standardization of [³H]-glucose uptake with insulin.

Table 6: Standardization of the optimal concentration of plant extract that retains cell viability by MTT assay

Table 7: Standardization of the time point of incubation of cells with the plant extract required in the assay while retaining cell viability by MTT assay

Table 8: [³H]-deoxyglucose uptake by the adipocytes from 3T3-L1 cells treated with plant extracts in the absence of insulin.

Table 9: [³H]-deoxyglucose uptake by adipocytes in presence of insulin, compared to the uptake observed in cells plus insulin alone

Table 10: MTT assay for extracts that are getting considered as potential lead candidates.

Table 11: The inhibitory activity of various concentrations of a plant extract in the alpha-glucosidase assay.

Table 12: List of plant extracts subject to successive extraction

Table 13: Validation of 3T3 assay

Table 14: Screening of successive extracts of Eugenia jambolana & Cinnamomum zeylanicum and the Counts per minute observed in study of insulin mimetic activity (without insulin) and study of insulin sensitizing activity (with insulin)

Table 15: Screening of successive extracts of Emblica officinalis & Catharanthus roseus and the Counts per minute observed in study of insulin mimetic activity (without insulin) and study of insulin sensitizing activity (with insulin)

### DETAILED DESCRIPTION OF THE INVENTION

We have screened plants as described in Table 1 using solvents as described in Table 2.

We have done both direct parallel and successive extraction for the plants as described in Table 1. We have screened a majority of the plant extracts for their efficiency against degenerative diseases. The present invention relates to the function of multiple plant extracts that have been validated for specific anti-diabetic usage using multiple etiology based cell assays. It also relates to any future preparation of any number of compositions for degenerative diseases like cardiovascular diseases, Diabetes mellitus, Hypertension, All types of Cancers, Obesity & weight problems, Arthritis & pain, Alzheimer's disease, Multiple sclerosis & Autoimmune diseases, Asthma & Allergies, Osteoporosis & bone health, Parkinson's disease, Stress, Disorders & diseases of the eye and having antioxidant action, a process for the preparation of the same, and usage of the same. The invention also relates, more particularly, to any composition having as a major component a single extract or multiple extracts containing therapeutically relevant bio-actives as characterized here in Tables 4, 8, 9, 10, 11 & 12, obtained from plant/plants as described in Tables 1, 2 and 3 and being capable of lowering or reducing a level of glucose in the blood of a patient suffering from diabetes mellitus by alleviating or ameliorating various symptoms caused by or associated with the diabetes mellitus as well as preventing the glucose level in the blood of the diabetic patient.

This application claims benefit of priority 35 U.S.C 119 to PCT application no. PCT/IB03/03861 filed on 18^{th} Aug. 2003 and is incorporated herein in its entirety by reference.

A comprehensive review of the literature and documented evidence concerning the usage of medicinal plants and plant parts in the treatment of degenerative disorders will result in a list of greater than 200 plant species that have been reported to have varying degrees of efficiency. A meaningful screening procedure that would involve all the plants documented to have antidegenerative potential will require a huge allocation of resources, which in turn would make the entire process unviable. To circumvent this problem, ADePt™ was used during the lead selection process to arrive at a smaller number of plants (less than 20) that are being subjected to bioactivity based screening procedures.

This information has allowed a rational selection of the plants and plant parts, which have been the immediate focus for the discovery of novel bio-therapeutics for the treatment of all degenerative diseases like cardiovascular diseases, Diabetes mellitus, Hypertension, All types of Cancers, Obesity & weight problems, Arthritis & pain, Alzheimer's disease, Multiple sclerosis & Autoimmune diseases, Asthma & Allergies, Osteoporosis & bone health, Parkinson's disease, Stress, Disorders & diseases of the eye.

The targeted screening procedures feature, a comprehensive metabolite profiling of multitudes of phyto-extracts facilitate the creation of a metabolite grid. Extensive comparative analyses of the individual plant species with the pre-existing drug and phyto-extract formulations in the market reveals the presence of both unique and common molecular constituents that can be used individually and in combination to accelerate the process of discovery of novel therapeutic formulations.

The extraction and resolution of components present in complex phyto-extracts is an area that has been plagued by a lack of standard operating procedures that permit adequate standardization and quantitative estimation of metabolites at a comprehensive level. As part of the ongoing research program at Avesthagen, standard operating protocols have now been setup for all the steps that precede the enzymatic and cell-based bioassay procedures that are aimed at the screening of the multi-component phyto-extracts.

In this context, algorithms that help to establish comprehensive metabolite fingerprints of multi-component phyto-extracts have been integrated into ADePt™. Currently, ADePt™ features metabolite fingerprints of approximately phyto-extracts, derived from 15 short-listed medicinal plants with documented anti-degenerative potential that are being screened for bioactive constituents. Following are the HPLC conditions used for the analysis different medicinal plant extracts.

Instrument: Waters 2695 Separation module with Millennium³² software.

Detector: Waters 2996 photodiode array.

Column: μBandapak C-18, 4.6×150 mm column, 10 μm particle size.

Solvent A: Acetonitrile

Solvent B: Methanol

Solvent C: Milli-Q water

The list of plants described in table 1 were subject to successive extraction, starting with Petroleum ether, followed by Benzene, Chlororform, Ethyl acetate, Acetone, Methanol and Water; as described by the fraction id nos. in Table 12.

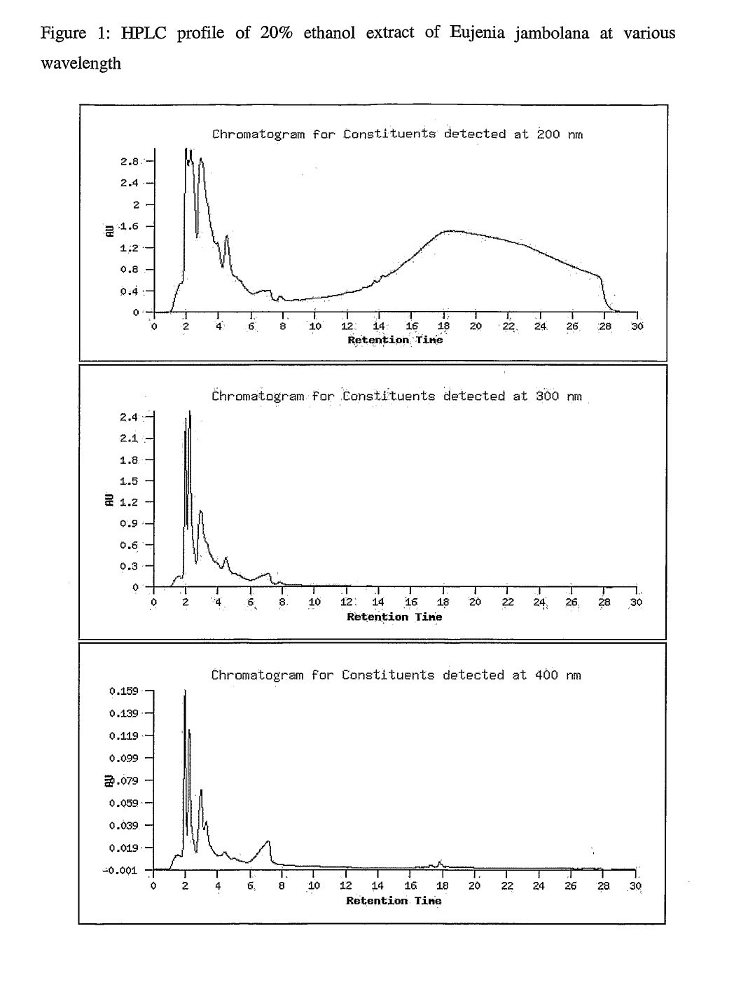

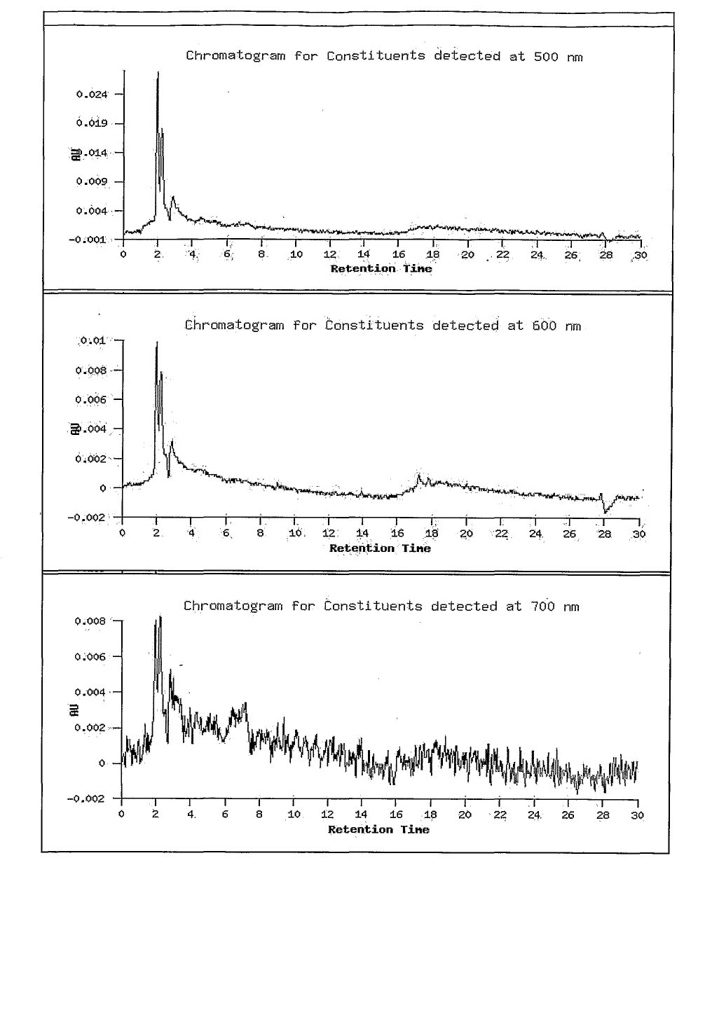

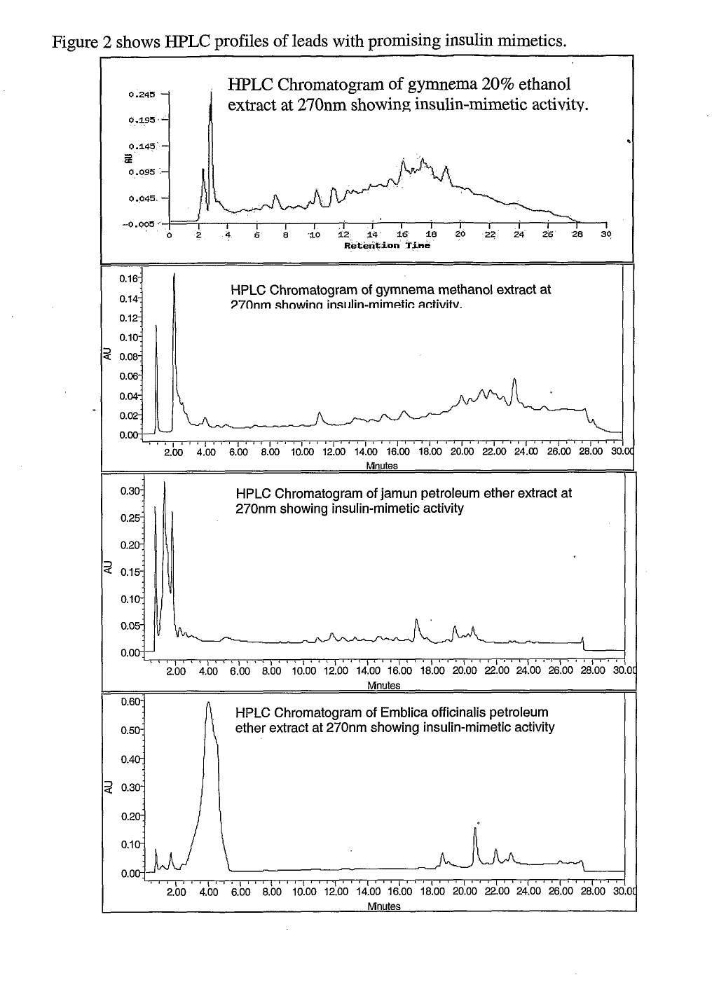

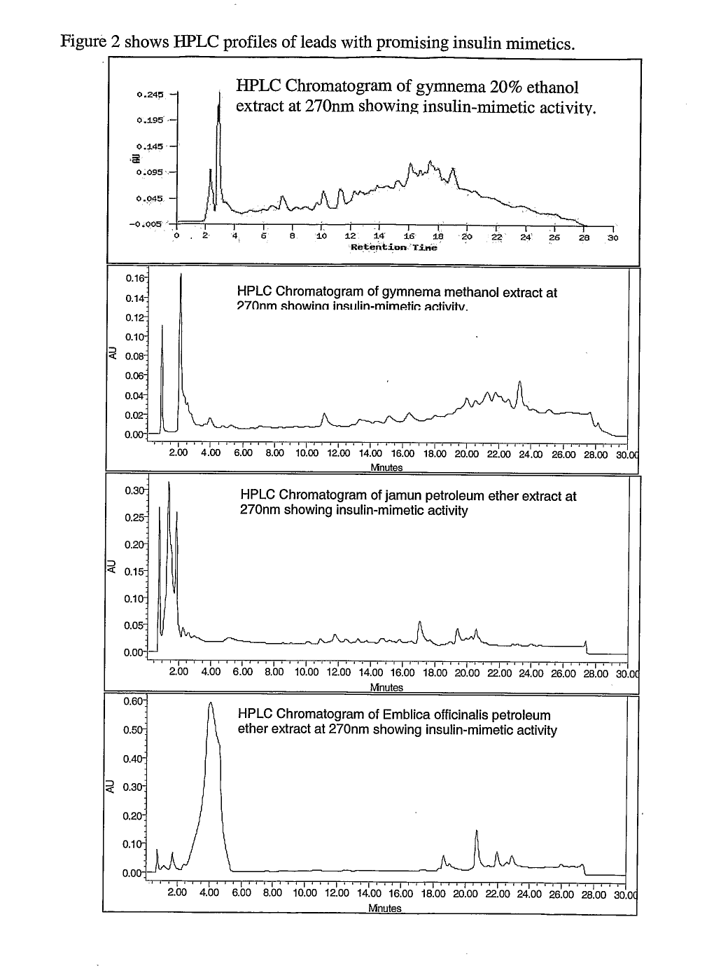

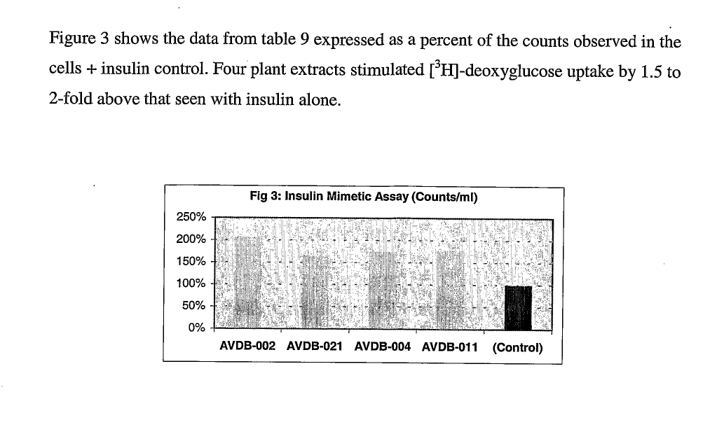

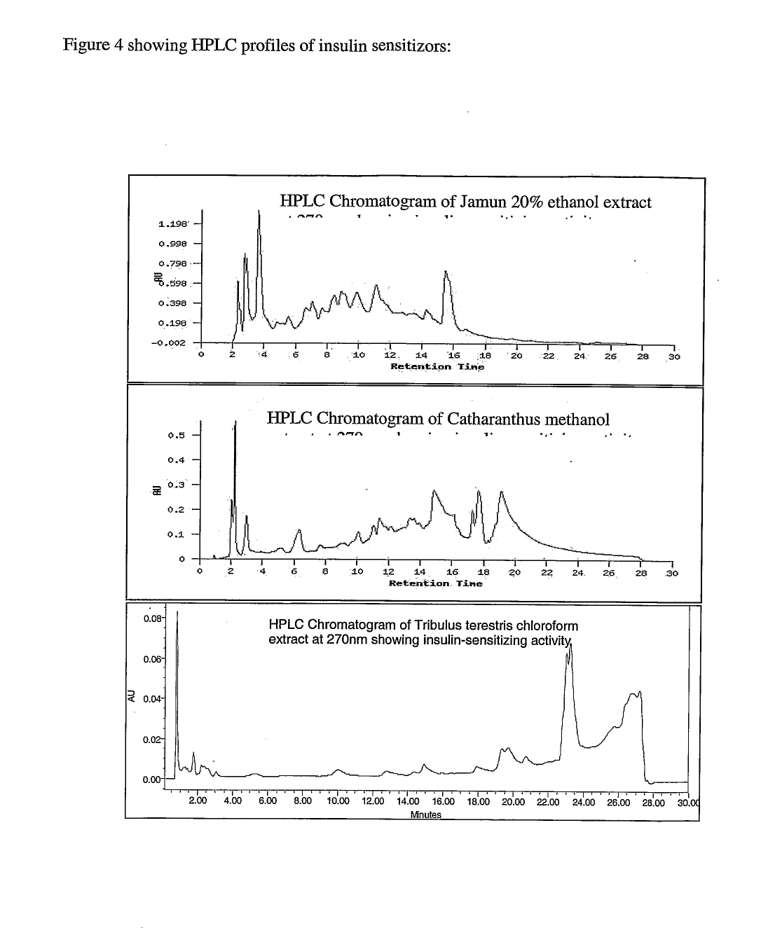

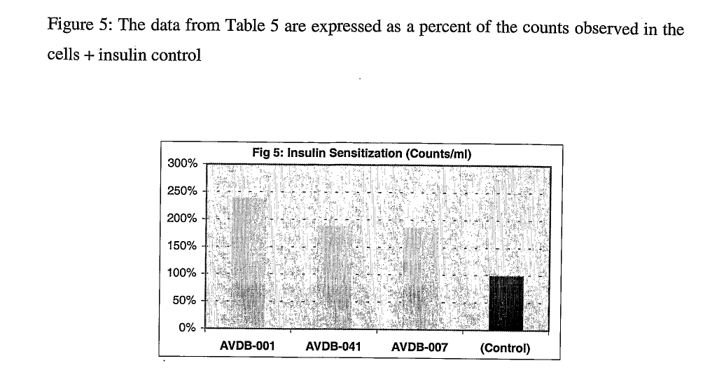

FIG. 1 shows an example of how an integrated metabolite fingerprinting is run for one 20% ethanol extract of Eugenia jambolana at various wavelengths

The basic investigational strategy for the search of an agent having anti-degenerative potential is based on either of the two indicatory factors for the disease i.e. insulin and glucose. Table 1 gives the list of plants which were used for the experiment, table 2 gives the list of solvents used for extraction, table 3 gives the plant part used for the extraction and Table 4 & Table 12 gives the list of extracts under experimentation. The cell-based assays selected to measure glucose metabolism are based on the following parameters: Glucose uptake and Glycogen synthesis

### EXAMPLES OF THE EFFICACY OF THE EXTRACTS

### Example 1

### Study of Insulin Mimetic Activity

“[³H]-deoxyglucose uptake by 3T3-L1 adipocytes” is the classic assay that measures the ability of bioactive lead candidates to improve insulin sensitivity in stimulation of glucose uptake by adipocytes. The assay can also be adapted to measure insulin-mimetic activity by measuring stimulation of glucose uptake by plant extracts in the absence of insulin.

In this study we are looking for molecules that have insulin mimetic activity. So we measured the uptake of 3H-de-oxyglucose in adipocytes by incubating them with plant extracts in the absence of insulin. Mature adipocytes derived from 3T3-L1 fibroblasts were incubated with plant extracts alone followed by [³H]-deoxyglucose uptake assay as described in details under the methods.

Method:

3T3-L1-Cell Culture

3T3-L1 fibroblasts were cultured in DME-F12 media containing 10% FCS, NEAA, glutamine, antibiotics, anti-mycotics, etc., in an atmosphere of 5% CO₂ at 37° C. Fibroblasts were cultured up to confluency. Subcultures were done at three-day intervals with trypsin-EDTA solution treatment.

Maturation of the Fibroblasts to Adipocytes

Differentiation was induced by treating the cells with DMEM hi glucose (4.5 gm/litre) containing 0.5 mmol/l 3-isobutyl-1-methylxanthine, 4 mg/ml dexamethasone, 1 mg/ml insulin and 10% FCS for 48 h. The cells were cultured in DMEM media with standard pre-described supplements for the next 6-10 days. Cells were used only when at least 95% of the cells showed an adipocyte phenotype by accumulation of lipid droplets.

3H-deoxyglucose Uptake

In the first set of assays (insulin control and the first 9 samples) cells were grown in 96 well plates, and assayed for 3H-deoxyglucose uptake. Briefly cells were treated with plant extract overnight and immediately before the assay got washed with Krebs-Ringer-HEPES Buffer [20 mM HEPES, pH 7.4, 136 mM NaCl, 4.7 mM KCl, 1.5 mM MgSO4] at 37° C. for 30 minutes and then treated with insulin. Glucose uptake reaction was initiated by adding 0.1 mM deoxy-glucose containing deoxy-tritiated-glucose containing 12.2 kBq/mL. After 30 minutes of 37° C. incubation, the reaction was terminated by adding 40 uM cytochalasin-B. Cells were washed 3× with ice cold KRH buffer and solubilized with 100 μl of 10% Triton-X with Ca/Mg free PBS. Radioactivity incorporated into cells was measured on a top count microplate scintillation counter. Results are being interpreted as mean±SD of counts per minute.

Table 5 shows standardization of [³H]-glucose uptake with insulin. As the basal counts were low, the assay design was changed to a 24-well format and the cell numbers increased from 100,000 cells per well to 2 million cells per well. As expected, this resulted in a dramatic increase in [³H] intake counts while maintaining a high level of sensitivity to insulin.

Table 8 shows [³H]-deoxyglucose uptake by the adipocytes from 3T3-L1 cells treated with plant extracts in the absence of insulin. The two controls in this experiment were: (a) [³H]-deoxyglucose uptake by cells in the absence of both insulin and plant extracts, and (b) [³H]-deoxyglucose uptake by cells in the presence of insulin and absence of plant extracts.

It was found that four plant extracts; namely AVDB008Le04(20)bc6C01A00, AVDB008Le06bc6C01A00, AVDB006Se07bc6C01A00 and AVDB005Le07bc6C01A00 stimulated [³H]-deoxyglucose uptake by 1.5 to 2-fold above that seen with insulin alone.

### Example 2

### Study of Cell Viability and Respiratory Physiology

Crude plant extracts solubilized in a “compatible” solvent such as dimethylsulfoxide (DMSO) often affect the viability of cells in culture when incubated for long periods. It is necessary to optimize the appropriate dilution and time of incubation of cells with plant extracts under which cell viability remains unaffected. The reduction of tetrazolium salts is now widely accepted as a reliable way to examine cell proliferation. The yellow tetrazolium salt, (MTT) 3-(4,5-dimethylthiazolyl-2)-2,5-diphenyltetrazolium bromide is reduced by metabolically active cells, in part by the action of dehydrogenase enzymes to generate reducing equivalents such as NADH and NADPH. The resulting intracellular purple formazan can be solubilized and quantified by spectrophotometric means.

MTT Assay to Determine Cell Viability when Incubated with Plant Extracts

Method:

3T3-L1-Cell Culture

3T3-L1 fibroblasts were cultured in DME-F12 media containing 10% FCS, NEAA, glutamine, antibiotics, anti-mycotics, etc., in an atmosphere of 5% CO₂ at 37° C. Fibroblasts were cultured up to confluency. Subcultures were done at three-day intervals with trypsin-EDTA solution treatment.

MTT Assay

The assay was done in 96 well flat bottom TC plates where the yellow tetrazolium MTT (3-(4,5-dimethylthiazolyl-2)-2,5-diphenyltetrazolium bromide) is added to the cells treated or untreated with the plant extracts. It gets reduced by metabolically active cells, in part by the action of dehydrogenase enzymes, to generate reducing equivalents such as NADH and NADPH. The resulting intracellular purple formazan can be solubilized and quantified by spectrophotometric means. For each cell type the linear relationship between cell number and signal produced is established, thus allowing an accurate quantification of changes in the rate of cell proliferation.

### Example 2a

### Study of Standardization of the Dilution of the Plant Extracts

3T3-L1 fibroblasts were incubated with different concentrations of stock concentration of plant extracts (neat—50 μg dry extract dissolved in 300 μl DMSO, and 1:2 and 1:4 dilutions of it) for 24 hrs. The viability of the cells was assessed by MTT cell assays. Table 6 shows that neat extracts gave maximal MTT colorimetric reading. The standardization study was done with selective extracts only.

Table 6 and 7 show the standardization of the optimal concentration of plant extract that retains cell viability by MT assay. 3T3-L1 fibroblasts were incubated with different concentrations of stock concentration of plant extracts (neat—50 μg dry extract dissolved in 300 μl DMSO, and 1:2 and 1:4 dilutions of it) for 24 hrs. The viability of the cells was assessed by MTT cell assays. Table 6 shows that neat extracts gave maximal MTT colorimetric reading. The standardization study was done with selective extracts only.

### Example 2b

### Study of Standardization of the Time Points of Co-Culture of the Extracts with the Cell Line

3T3-L1 fibroblasts were incubated with stock concentration of plant extracts (neat—50 μg dry extract dissolved in 300 μl DMSO) for 24, 48 and 72 hrs. The viability of the cells was assessed by MTT cell assays. Table 7 describes the standardization of the time point of incubation of cells with the plant extract required in the assay while retaining cell viability by MTT assay. It shows that the optimal incubation time for most of the extracts was 24 hrs. This standardization study was also done with selective extracts only. The routine MTT assay was done on all extract fractions and extracts that showed no cytotoxicity were taken up for evaluation of 3H de-oxyglucose uptake test.

### Example 2c

### Study of the Influence of the Extracts in Producing Respiratory Enzymes in the Cell Line

3T3-L1 fibroblasts were incubated with stock concentration of plant extracts (neat—50 μg dry extract dissolved in 300 μl DMSO) for 24. Table 10 shows the optical density of the cell lysate that produced the formazon crystals. The color intensity corresponded with the amount of formazon produced that is an indicator of respiratory enzymes generated.

### Example 3

### Study of Insulin Sensitizing Activity

In this study stimulation of [³H]-deoxyglucose uptake in adipocytes was measured by incubating them with plant extracts in the presence of insulin. Mature adipocytes derived from 3T3-L1 fibroblasts were incubated with plant extracts in the presence of insulin followed by [³H]-deoxyglucose uptake assay as described in details under the methods of example 1.

Table 9 and FIGS. 4 and 5 shows the HPLC profile and activity of the three plant extracts enhanced [³H]-deoxyglucose uptake by adipocytes in presence of insulin, compared to the uptake observed in cells plus insulin alone. Table 4 below shows the raw counts.

In FIG. 5, below, the data from Table 5 are expressed as a percent of the counts observed in the cells+insulin control.

It is observed that three plant extracts; namely, AVDB006Se04(20)bc6C01A00, AVDB018Fr03bc6C01A00 & AVDB003Ro06bc6C01A00, stimulated [³H] deoxyglucose uptake by nearly 2-fold above that seen with insulin alone. This enhancement in uptake of [³H]-deoxyglucose in the presence of insulin indicates that the plant extract activity results in increased sensitization of adipocytes to respond to insulin.

### Example 4

### Alpha-Glucosidase Inhibition Assay

Postprandial hyperglycemia and hyperinsulinemia, which are implicated in Diabetes and Obese conditions, are expected to be diminished by the inhibition of poly and oligosaccharide digestion in the intestinal tract. Alpha-glucosidase inhibitors, which act as competitive inhibitors of intestinal alpha-glucosidases, can delay the digestion and subsequent absorption of blood glucose rises.

Method

Various concentrations of plant extracts (inhibitor) are pre-incubated with the enzyme before adding the substrate (PNPG). Alpha-Glucosidase activity can be measured in-vitro by determination of the release of p-nitrophenol arising from the hydrolysis of p-nitrophenyl-alpha-D-glucopyranoside by alpha-Glucosidase. The colour developed by the release of PNP (p-nitro phenol) can be measured quantitatively in the spectrophotometer.

Several experiments were performed in which the concentrations of a-glucosidase at constant substrate concentration was varied and the concentration of substrate was varied at constant substrate concentrations. An enzyme concentration of 0.04 U/ml and the substrate (PNPG) concentration of 4 mM were selected for the screening assays.

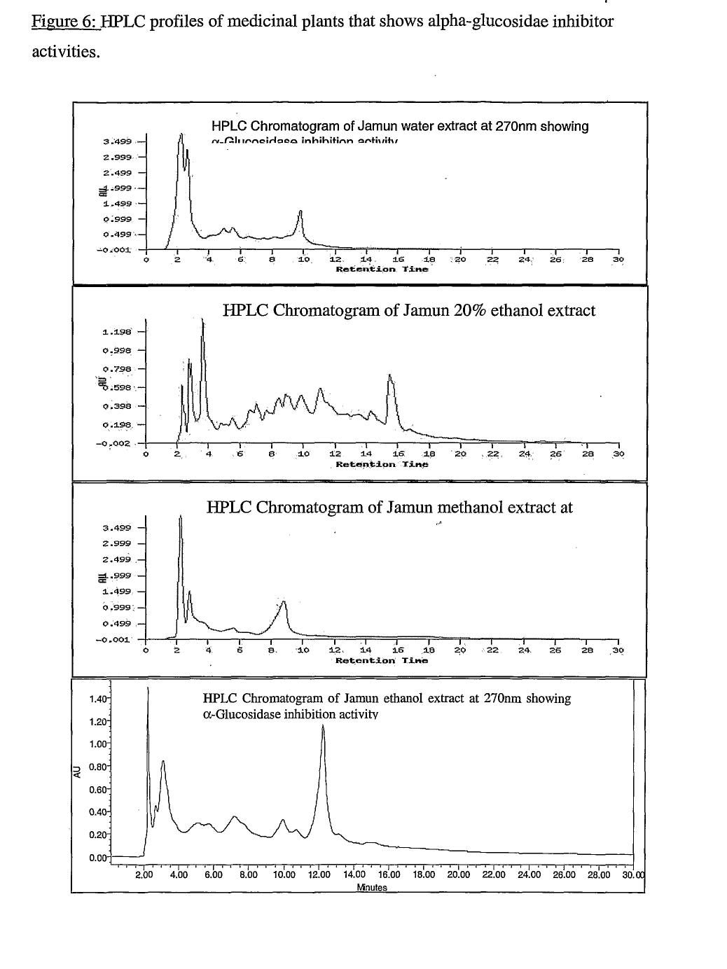

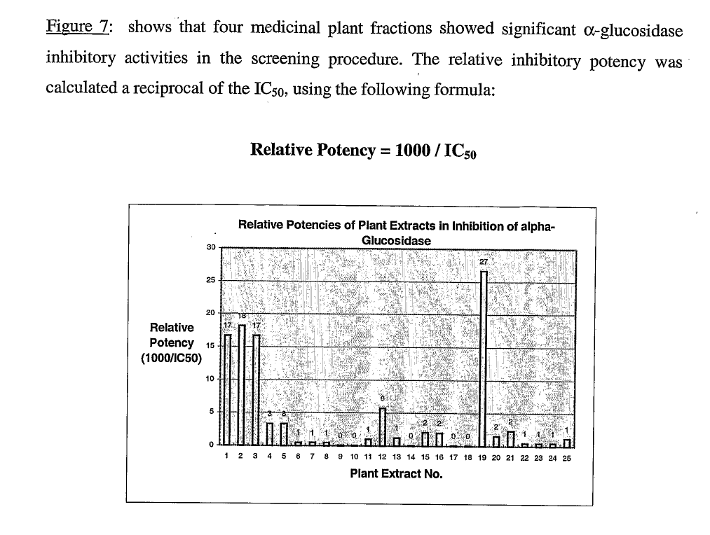

Table 11 & FIGS. 6 and 7 shows the inhibitory activity of various concentrations of a plant extract in the alpha-glucosidase assay. In this case the IC₅₀ (concentration showing 50% inhibition) was determined to be 50 μg/ml.

It was found that four medicinal plant extracts AVDB006Se08bc6C01A00, AVDB006Se04(20)bc6C01A00, AVDB006Se06bc6C01A00 and AVDB006Se04bc6C01A00 showed significant alpha-glucosidase inhibitory activity in the screening procedure.

While the present invention has been particularly shown and described with reference to the preferred embodiment thereof, it will be understood by those skilled in the art that various changes in form and details may be effected therein without departing from the spirit and scope of the invention as defined by the appended claims.

TABLE 1
List of plants and their codes

| Sr. No. | Plant Name | Plant ID |
|---|---|---|
| 1 | Aegle marmelos | 001 |
| 2 | Azadirachta indica | 002 |
| 3 | Catharanthus roseus | 003 |
| 4 | Curcuma longa | 004 |
| 5 | Emblica officinalis | 005 |
| 6 | Eugenia jambolana | 006 |
| 7 | Glycyrrhiza glabara | 007 |
| 8 | Gymnema sylvestre | 008 |
| 9 | Lagerstroemia speciosa | 009 |
| 10 | Momordica charantia | 010 |
| 11 | Morinda citrifolia | 011 |
| 12 | Ocimum sanctum | 012 |
| 13 | Pterocarpus marsupium | 013 |
| 14 | Stevia rebaudiana | 014 |
| 15 | Swertia chirata | 015 |
| 17 | Tinospora cordifolia | 017 |
| 18 | Tribulus terestris | 018 |
| 19 | Trigonella foenum gracum | 019 |
| 20 | Withania somnifera | 020 |
| 21 | Cinnamomum zeylanicum | 021 |
| 22 | Garcinia combogia | 022 |
| 23 | Camellia sinensis | 023 |
| 24 | Vanilla fragrans | 024 |
| 25 | Coccinia indica | 025 |

TABLE 2
Table of Solvents used in the experiments and their codes

| Solvent name | Solvent ID |
|---|---|
| Acetone | 01 |
| Benzene | 02 |
| Chloroform | 03 |
| Ethanol | 04 |
| Hexane | 05 |
| Methanol | 06 |
| Petroleum ether | 07 |
| Water | 08 |
| Ethyl acetate | 09 |
| Acetonitrile | 10 |

TABLE 3
Table of Plant part/tissue used in the experiment and their codes

| | Part/Tissue |
|---|---|
| Plant Part/Tissue | ID |
| Aerial Parts | AP |
| Bark | Ba |
| Flower | Fl |
| Fruit Whole | Fr |
| Fruit pulp | FP |
| Leaf | Lf |
| Pod | Po |
| Rhizome | Rh |
| Root | Ro |
| Seed | Se |
| Tuber | Tu |
| Whole Plant | WP |

TABLE 4
List of fractions of the plant extracts subjected to cell assays and the
Counts per minute observed in study of insulin mimetic activity
(without insulin) and study of insulin sensitizing activity (with insulin)

| | | CPM | |
|---|---|---|---|
| Sample | | without | CPM with |
| number | Fraction ID | Insulin | Insulin |
| 1 | AVDB006Se08bc6C01A00 | 3104.8 | 59317.3 |
| 2 | AVDB006Se04(20)bc6C01A00 | 50002.9 | 90587.7 |
| 3 | AVDB019Se08bc6C01A00 | 32995.3 | 40797.1 |
| 4 | AVDB019Se04(20)bc6C01A00 | 35818 | 26256.8 |
| 6 | AVDB008Le08bc6C01A00 | 49174.8 | 1836.9 |
| 7 | AVDB008Le04(20)bc6C01A00 | 78730 | 41782.6 |
| 8 | AVDB013Ba08bc6C01A00 | 39701.8 | 43449.2 |
| 9 | AVDB013Ba04(20)bc6C01A00 | 40732.3 | 35926 |
| 10 | AVDB002Ba08bc6C01A00 | 40406.1 | 41058.9 |
| 11 | AVDB005Fr06bc6C01A00 | 54150.5 | 2718.5 |
| 12 | AVDB003Ro06bc6C01A00 | 40569.8 | 71661.7 |
| 13 | AVDB020Ro06bc6C01A00 | 51048.2 | 10421.6 |
| 14 | AVDB019Se06bc6C01A00 | 41022 | 4662.6 |
| 15 | AVDB006Se06bc6C01A00 | 45698.5 | 4318.3 |
| 16 | AVDB002Le06bc6C01A00 | 29289.9 | 16257.4 |
| 17 | AVDB018Fr06bc6C01A00 | 33852.6 | 3196.3 |
| 18 | AVDB017Fr06bc6C01A00 | 45025.2 | 2564.8 |
| 19 | AVDB003Ro03bc6C01A00 | 44878.3 | 380.4 |
| 20 | AVDB020Ro04bc6C01A00 | 35140.5 | 11758.2 |
| 21 | AVDB020Ro03bc6C01A00 | 25536.6 | 38342.5 |
| 22 | AVDB019Seo04bc6C01A00 | 34922 | 31443.8 |
| 23 | AVDB004Se04bc6C01A00 | 40097.2 | 54145.4 |
| 24 | AVDB018Fr04bc6C01A00 | 5030.4 | 35612.5 |
| 25 | AVDB011Fr04bc6C01A00 | 21884.3 | 22019.8 |
| 26 | AVDB017Fr04bc6C01A00 | 30027.6 | 22528.9 |
| 27 | AVDB019Se03bc6C01A00 | 32921.9 | 32578.4 |
| 28 | AVDB019Se03bc6C01A00 | 36945 | 50268.6 |
| 29 | AVDB006Se03bc6C01A00 | 8765.5 | 31362.3 |
| 30 | AVDB011Fr03bc6C01A00 | 39609.9 | 29006.6 |
| 31 | AVDB017Fr03bc6C01A00 | 43817.2 | 26515.9 |
| 32 | AVDB005Fr03bc6C01A00 | 42664.5 | 46780.6 |
| 33 | AVDB008Le06bc6C01A00 | 41279.6 | 747.7 |
| 34 | AVDB006Fr03bc6C01A00 | 689.1 | 41018.5 |
| 35 | AVDB020Ro07bc6C01A00 | 2475.9 | 20976.4 |
| 36 | AVDB019Se07bc6C01A00 | 3198.7 | 24380.4 |
| 37 | AVDB006Se07bc6C01A00 | 67493 | 21301.2 |
| 38 | AVDB018Fr07bc6C01A00 | 3572.9 | 4940.8 |
| 39 | AVDB008Le07bc6C01A00 | 30539.8 | 62764.2 |
| 40 | AVDB002Le05bc6C01A00 | 2594.6 | 28254.4 |
| 41 | AVDB020Ro02bc6C01A00 | 2539.8 | 10946.4 |
| 42 | AVDB019Se02bc6C01A00 | 30864 | 32429.1 |
| 43 | AVDB002Se02bc6C01A00 | 3750 | 36523.4 |
| 44 | AVDB018Fr02bc6C01A00 | 3103.1 | 20013.5 |
| 45 | AVDB008Le06bc6C01A00 | 62200 | 32112.7 |
| 46 | AVDB002Le02bc6C01A00 | 2185 | 44102 |
| 47 | AVDB020Le08bc6C01A00 | 89.2 | 26084.7 |
| 48 | AVDB008Le04bc6C01A00 | 1651.6 | 24005 |
| 49 | AVDB002Le03bc6C01A00 | 50451.2 | 27349.1 |
| 50 | AVDB002Le04bc6C01A00 | 1128.7 | 37275.8 |
| 51 | AVDB002Le07bc6C01A00 | 546.5 | 64153.8 |
| 52 | AVDB018Fr03bc6C01A00 | 16783.8 | 70859.9 |
| 53 | AVDB008Le02bc6C01A00 | 12940 | 54596.2 |
| 54 | AVDB003Ro04bc6C01A00 | 1953.6 | 30342.6 |
| 55 | AVDB008Le03bc6C01A00 | 16799.2 | 47831.4 |
| 56 | AVDB013Ba04bc6C01A00 | 17385.3 | 22041.3 |
| 57 | AVDB008Le09bc6C01A00 | 24436.1 | 56640.7 |
| 58 | AVDB008Le01bc6C01A00 | 37726.5 | 32916.7 |
| 59 | AVDB019Se09bc6C01A00 | 35199 | 33672.2 |
| 60 | AVDB019Se01bc6C01A00 | 59363.1 | 38863.4 |
| 61 | AVDB005Le07bc6C01A00 | 66332.8 | 28386.2 |
| 62 | AVDB005Le05bc6C01A00 | 33880.3 | 52381.6 |
| 63 | AVDB006Se05bc6C01A00 | 44015.3 | 7996.3 |

TABLE 5
Table showing standardization of [³H]-glucose uptake with insulin.

| | | | | Insulin | Insulin |
|---|---|---|---|---|---|
| Cells/well | Control | Insulin 1 nM | Insulin 10 nM | 25 nM | 100 nM |
| 100,000 | 25.3 | 60.5 | 502 | ND | 1850 |
| 2,000,000 | 1559 | ND | 36562 | 38538 | 55258 |

TABLE 6
Standardization of the optimal concentration of plant extract that retains
cell viability by MTT assay

| | Fr no. | | | | | | | | |
|---|---|---|---|---|---|---|---|---|---|
| Dil | 1 | 2 | 3 | 4 | 5 | 6 | 7 | 8 | 9 |
| Neat | .811* | 1.188 | .411 | .412 | .376 | .311 | .717 | .698 | .523 |
| 1:2 | .628 | .726 | .323 | .363 | .372 | .279 | .457 | .516 | .547 |
| 1:4 | .482 | .519 | .390 | .304 | .356 | .301 | .479 | .396 | .428 |

TABLE 7
Standardization of the time point of incubation of cells with the plant
extract required in the assay while retaining cell viability by MTT assay

| | Fr no. | | | | | | | | |
|---|---|---|---|---|---|---|---|---|---|
| Dil | 1 | 2 | 3 | 4 | 5 | 6 | 7 | 8 | 9 |
| 24 hrs | .811* | 1.188 | .411 | .412 | .376 | .311 | .717 | .698 | .523 |
| 48 hrs | .534 | 1.262 | .332 | .270 | .304 | .288 | .525 | .427 | .375 |
| 72 hrs | .142 | 1.199 | .218 | .223 | .287 | .267 | .392 | .298 | .403 |

*Optical density (O.D. at 490 nm) control values - a) fibroblast alone 0.195 and b) DMSO control 0.285.

TABLE 8
[³H]-deoxyglucose uptake by the adipocytes from 3T3-L1 cells
treated with plant extracts in the absence of insulin. (mimetic)

| | | Counts |
|---|---|---|
| Sr. No. | Extract ID. | per minute |
| 1 | AVDB008Le04(20)bc6C01A00 | 78730 |
| 2 | AVDB008Le04(20)bc6C01A00 | 66332 |
| 3 | AVDB006Se07bc6C01A00 | 67493 |
| 4 | AVDB008Le06bc6C01A00 | 62200 |
| 5 | Cells alone | 1559 |
| 6 | Cells + insulin | 36561 |

TABLE 9
[³H]-deoxyglucose uptake by adipocytes in presence of insulin,
compared to the uptake observed in cells plus insulin alone.

| | | Counts |
|---|---|---|
| Sr. No. | Extract ID | per minute |
| 1 | AVDB006Se04(20)bc6C01A00 | 90587 |
| 2 | AVDB018Fr03bc6C01A00 | 70859 |
| 3 | AVDB003Ro06bc6C01A00 | 71661 |
| 4 | Cells alone | 1559 |
| 5 | Cells + insulin | 36561 |

TABLE 10
MTT assay:

| Extract nos | O.D. at 490 nm |
|---|---|
| AVDB008Le04(20)bc6C01A00 | 1.188 |
| AVDB008Le04(20)bc6C01A00 | 0.311 |
| AVDB006Se07bc6C01A00 | 0.414 |
| AVDB008Le06bc6C01A00 | 0.497 |
| AVDB006Se04(20)bc6C01A00 | 0.087 |
| AVDB018Fr03bc6C01A00 | 0.08 |
| AVDB003Ro06bc6C01A00 | 0.131 |

TABLE 11
The inhibitory activity of various concentrations of a plant extract in the
alpha glucosidase assay

| No. | Extract ID | IC₅₀/Potency | | | 1/IC₅₀ |
|---|---|---|---|---|---|
| 1 | AVDB006Se08bc6C01A00 | 60 | μg/ml | +++ | 17 |
| 2 | AVDB006Se04(20)bc6C01A00 | 55 | μg/ml | +++ | 18 |
| 3 | AVDB006Se06bc6C01A00 | 60 | μg/ml | +++ | 17 |
| 4 | AVDB013Ba08bc6C01A00 | 300 | μg/ml | ++ | 3 |
| 5 | AVDB013Ba08bc6C01A00 | 300 | μg/ml | ++ | 3 |
| 6 | AVDB019(20)Se02bc6C01A00 | >1000 | μg/ml | − | 1 |
| 7 | AVDB019Se08bc6C01A00 | >1000 | μg/ml | − | 1 |
| 8 | AVDB008Le04(20)bc6C01A00 | >1000 | μg/ml | − | 1 |
| 9 | AVDB008Le08bc6C01A00 | No activity | | − | 0 |
| 10 | AVDB020Ro08bc6C01A00 | No activity | | − | 0 |
| 11 | AVDB020Ro04bc6C01A00 | 1000 | μg/ml | + | 1 |
| 12 | AVDB002Le08bc6C01A00 | 175 | μg/ml | ++ | 6 |
| 13 | AVDB017Fr04bc6C01A00 | 800 | μg/ml | + | 1 |
| 14 | AVDB003Ro04bc6C01A00 | No activity | | − | 0 |
| 15 | AVDB011Fr04bc6C01A00 | 475 | μg/ml | + | 2 |
| 16 | AVDB018Fr04bc6C01A00 | 500 | μg/ml | ++ | 2 |
| 17 | AVDB019Se04bc6C01A00 | No activity | | − | 0 |
| 18 | AVDB008Le04bc6C01A00 | No activity | | − | 0 |
| 19 | AVDB006Se04bc6C01A00 | 37.5 | μg/ml | +++ | 27 |
| 20 | AVDB002Le04bc6C01A00 | 650 | μg/ml | + | 2 |
| 21 | AVDB005Fr06bc6C01A00 | 425 | μg/ml | ++ | 2 |
| 22 | AVDB003Ro06bc6C01A00 | >1000 | μg/ml | − | 1 |
| 23 | AVDB020Ro06bc6C01a00 | >1000 | μg/ml | − | 1 |
| 24 | AVDB019Se06bc6C01A00 | >1000 | μg/ml | − | 1 |
| 25 | AVDB011Fr06bc6C01A00 | 850 | μg/ml | + | 1 |

TABLE 12
List of plant extracts subject to successive extraction

| Sr. | |
|---|---|
| No. | Nomenclature |
| 1 | AVDB008Lf7S07(100)A1 |
| 2 | AVDB008Lf7S02(100)A1 |
| 3 | AVDB008Lf7S03(100)A1 |
| 4 | AVDB008Lf7S09(100)A1 |
| 5 | AVDB008Lf7S01(100)A1 |
| 6 | AVDB008Lf7S06(100)A1 |
| 7 | AVDB008Lf7S08(100)A1 |
| 8 | AVDB019Se7S07(100)A1 |
| 9 | AVDB019Se7S02(100)A1 |
| 10 | AVDB019Se7S03(100)A1 |
| 11 | AVDB019Se7S09(100)A1 |
| 12 | AVDB019Se7S01(100)A1 |
| 13 | AVDB019Se7S06(100)A1 |
| 14 | AVDB006Fp7S07(100)A1 |
| 15 | AVDB006Fp7S05(100)A1 |
| 16 | AVDB006Fp7S03(100)A1 |
| 17 | AVDB006Fp7S09(100)A1 |
| 18 | AVDB006Fp7S01(100)A1 |
| 19 | AVDB006Fp7S06(100)A1 |
| 20 | AVDB006Fp7S08(100)A1 |
| 21 | AVDB005Fr7S07(100)A1 |
| 22 | AVDB005Fr7S05(100)A1 |
| 23 | AVDB005Fr7S03(100)A1 |
| 24 | AVDB005Fr7S09(100)A1 |
| 25 | AVDB005Fr7S01(100)A1 |
| 26 | AVDB005Fr7S06(100)A1 |
| 27 | AVDB005Fr7S08(100)A1 |
| 28 | AVDB001Lf7S07(100)A1 |
| 29 | AVDB001Lf7S05(100)A1 |
| 30 | AVDB001Lf7S03(100)A1 |
| 31 | AVDB001Lf7S09(100)A1 |
| 32 | AVDB001Lf7S01(100)A1 |
| 33 | AVDB001Lf7S06(100)A1 |
| 34 | AVDB001Lf7S08(100)A1 |
| 35 | AVDB010Fr7S07(100)A1 |
| 36 | AVDB010Fr7S05(100)A1 |
| 37 | AVDB010Fr7S03(100)A1 |
| 38 | AVDB010Fr7S09(100)A1 |
| 39 | AVDB010Fr7S01(100)A1 |
| 40 | AVDB010Fr7S06(100)A1 |
| 41 | AVDB010Fr7S08(100)A1 |
| 42 | AVDB003Le7S07(100)A1 |
| 43 | AVDB003Le7S05(100)A1 |
| 44 | AVDB003Le7S03(100)A1 |
| 45 | AVDB003Le7S09(100)A1 |
| 46 | AVDB003Le7S01(100)A1 |
| 47 | AVDB003Le7S06(100)A1 |
| 48 | AVDB003Le7S08(100)A1 |
| 49 | AVDB012Ap7S07(100)A1 |
| 50 | AVDB012Ap7S05(100)A1 |
| 51 | AVDB012Ap7S03(100)A1 |
| 52 | AVDB012Ap7S09(100)A1 |
| 53 | AVDB012Ap7S01(100)A1 |
| 54 | AVDB012Ap7S06(100)A1 |
| 55 | AVDB012Ap7S08(100)A1 |
| 56 | AVDB013Ba7S07(100)A1 |
| 57 | AVDB013Ba7S05(100)A1 |
| 58 | AVDB013Ba7S03(100)A1 |
| 59 | AVDB013Ba7S09(100)A1 |
| 60 | AVDB013Ba7S01(100)A1 |
| 61 | AVDB013Ba7S06(100)A1 |
| 62 | AVDB013Ba7S08(100)A1 |
| 63 | AVDB014Lf7S07(100)A1 |
| 64 | AVDB014Lf7S05(100)A1 |
| 65 | AVDB014Lf7S03(100)A1 |
| 66 | AVDB014Lf7S09(100)A1 |
| 67 | AVDB014Lf7S01(100)A1 |
| 68 | AVDB014Lf7S06(100)A1 |
| 69 | AVDB014Lf7S08(100)A1 |
| 70 | AVDB018Wp7S07(100)A1 |
| 71 | AVDB018Wp7S05(100)A1 |
| 72 | AVDB018Wp7S03(100)A1 |
| 73 | AVDB018Wp7S09(100)A1 |
| 74 | AVDB018Wp7S01(100)A1 |
| 75 | AVDB018Wp7S06(100)A1 |
| 76 | AVDB018Wp7S08(100)A1 |
| 77 | AVDB010Fr7S07(100)A2 |
| 78 | AVDB010Fr7S05(100)A2 |
| 79 | AVDB010Fr7S03(100)A2 |
| 80 | AVDB010Fr7S09(100)A2 |
| 81 | AVDB010Fr7S01(100)A2 |
| 82 | AVDB010Fr7S06(100)A2 |
| 83 | AVDB010Fr7S08(100)A2 |

TABLE 13
Validation of 3T3-L1 assay

| Validation | | | Counts |
|---|---|---|---|
| molecules | Doses | Incubation Time | per minute |
| None | — | — | 4622.34 |
| Insulin | 25 nM | 15 min | 48026.7 |
| Insulin | 25 nM | 30 min | 24699.9 |
| Insulin | 25 nM | 60 min | 28103.3 |
| Glitazone | 60 μg | 48 hrs | 7886 |
| Glitazone | 150 μg | 48 hrs | 15900.4 |
| Glitazone | 300 μg | 48 hrs | 47971.9 |
| Glitazone + insulin | 60 μg + 25 nM | 48 hrs/15 min | 19590.5 |
| Glitazone + insulin | 150 μg + 25 nM | 48 hrs/15 min | 113168 |

TABLE 14
Screening of successive extracts and the Counts per minute observed in
study of insulin mimetic activity (without insulin) and study of insulin
sensitizing activity (with insulin)

| Extracts | Counts without insulin | Counts with insulin |
|---|---|---|
| None | 4622.34 | — |
| AVDB021Ba08bc6C01A00 | 16475.2 | 53583 |
| AVDB006Fp7S07(100)A1 | 11983.6 | 21189.5 |
| AVDB006Fp7S05(100)A1 | 23563.7 | 36229.8 |
| AVDB006Fp7S03(100)A1 | 31149.3 | 39991.6 |
| AVDB006Fp7S09(100)A1 | 36229.8 | 40132.4 |
| AVDB006Fp7S01(100)A1 | 413365 | 69396.0 |
| AVDB006Fp7S06(100)A1 | 28102.8 | 39429.8 |
| AVDB006Fp7S08(100)A1 | 13292.3 | 19981.9 |

TABLE 15
Screening of successive extracts and the Counts per minute observed in
study of insulin mimetic activity (without insulin) and study of insulin
sensitizing activity (with insulin)

| | Counts | | | |
|---|---|---|---|---|
| Extracts | without insulin | | Counts with insulin | |
| None | 1975.1 | 1831.0 | — | — |
| Insulin | — | — | 33065.1 | 32352.28 |
| AVDB005Fr7S07(100)A1 | 67490.1 | 66563.6 | 39933.1 | 38480.9 |
| AVDB005Fr7S05(100)A1 | 39196.9 | 38197.0 | 52662.1 | 57709.2 |
| AVDB005Fr7S03(100)A1 | 28311.7 | 13361.3 | 36229.8 | 39198.9 |
| AVDB005Fr7S09(100)A1 | 9055.6 | 8425.7 | 37506 | 37269 |
| AVDB005Fr7S01(100)A1 | 6671.7 | 6961.4 | 44696.2 | 42544.7 |
| AVDB005Fr7S06(100)A1 | 2400.5 | 2716.5 | 34279 | 32727 |
| AVDB005Fr7S08(100)A1 | 785.7 | 1011.4 | 39929 | 480959 |
| AVDB003Le7S01(100)A1 | 40580.3 | 48645.2 | 60119 | 73759 |
| AVDB003Le7S06(100)A1 | 28311.8 | 28205.2 | 31401.2 | 32590 |
| AVDB003Le7S08(100)A1 | 8294.9 | 7622.6 | 39929 | 38096 |

TABLE 16
HPLC method used for the analysis of AVDB006Se04bc6C01A00,
AVDB006Se04(20)bc6C01A00, AVDB006Se06bc6C01A00,
AVDB008Le04(20)bc6C01A00, AVDB008Le06bc6C01A00 and
AVDB003Ro06bc6C01A00.

| | |
|---|---|
| | Solvent |
| Flow | percentage |

| Time | (ml/min) | A | B | C | D | Curve |
|---|---|---|---|---|---|---|
| 1 | 1.0 | 10 | 10 | 80 | 0 | 6 |
| 5 | 1.0 | 10 | 25 | 65 | 0 | 6 |
| 10 | 1.0 | 10 | 50 | 40 | 0 | 6 |
| 15 | 1.0 | 10 | 90 | 0 | 0 | 6 |
| 20 | 1.0 | 10 | 80 | 10 | 0 | 6 |
| 25 | 1.0 | 10 | 60 | 30 | 0 | 6 |
| 26 | 1.0 | 10 | 10 | 80 | 0 | 1 |

TABLE 17
HPLC method was used for the analysis of AVDB006Se08bc6C01A00

| | |
|---|---|
| | Solvent |
| Flow | percentage |

| Time | (ml/min) | A | B | C | D | Curve |
|---|---|---|---|---|---|---|
| 1 | 1.0 | 10 | 0 | 90 | 0 | 6 |
| 5 | 1.0 | 15 | 0 | 85 | 0 | 6 |
| 10 | 1.0 | 20 | 0 | 80 | 0 | 6 |
| 15 | 1.0 | 25 | 0 | 75 | 0 | 6 |
| 20 | 1.0 | 35 | 0 | 65 | 0 | 6 |
| 25 | 1.0 | 60 | 0 | 40 | 0 | 6 |
| 26 | 1.0 | 10 | 0 | 90 | 0 | 1 |

TABLE 18
HPLC method used for the analysis of AVDB006Se07bc6C01A00

| | |
|---|---|
| | Solvent |
| Flow | percentage |

| Time | (ml/min) | A | B | C | D | Curve |
|---|---|---|---|---|---|---|
| 1 | 1.2 | 10 | 10 | 80 | 0 | 6 |
| 15 | 1.2 | 50 | 10 | 40 | 0 | 6 |
| 25 | 1.2 | 90 | 10 | 0 | 0 | 6 |
| 30 | 1.2 | 10 | 10 | 80 | 0 | 1 |

TABLE 18
HPLC method was used for the analysis of AVDB005Fr07bc6C01A00

| | |
|---|---|
| | Solvent |
| Flow | percentage |

| Time | (ml/min) | A | B | C | D | Curve |
|---|---|---|---|---|---|---|
| 1 | 1.2 | 10 | 20 | 70 | 0 | 6 |
| 15 | 1.2 | 50 | 20 | 30 | 0 | 6 |
| 25 | 1.2 | 80 | 20 | 0 | 0 | 6 |
| 30 | 1.2 | 10 | 20 | 70 | 0 | 1 |

TABLE 19
HPLC method used for the analysis of AVDB018Fr03bc6C01A00.

| | |
|---|---|
| | Solvent |
| Flow | percentage |

| Time | (ml/min) | A | B | C | D | Curve |
|---|---|---|---|---|---|---|
| 1 | 1.2 | 10 | 20 | 70 | 0 | 6 |
| 15 | 1.2 | 10 | 50 | 40 | 0 | 6 |
| 25 | 1.2 | 10 | 90 | 0 | 0 | 6 |
| 30 | 1.2 | 10 | 10 | 80 | 0 | 1 |

1-85. (canceled)

86. A plant extract demonstrating at least one of a) insulin mimetic activity, b) insulin sensitizing activity, c) alpha glucosidase activity, d) thermogenesis activity, e) enhancing glucose uptake activity by fat cells or peripheral skeletal muscle cells, f) decreasing glucose secretion by hepatocytes at liver, or g) increasing insulin secretion by pancreatic beta cells, having HPLC Fraction ID Nos. as identified in Table 4 or 8 to 11.

87. The plant extract of claim 86, having HPLC Fraction ID Nos. as identified in Table 4.

88. The plant extract of claim 86, having HPLC Fraction ID Nos. as identified in Tables 8 to 11.

89. A composition comprising an antioxidant effective amount of at least one plant extract demonstrating at least one of a) insulin mimetic activity, b) insulin sensitizing activity, c) alpha glucosidase activity, d) thermogenesis activity, e) enhancing glucose uptake activity by fat cells or peripheral skeletal muscle cells, f) decreasing glucose secretion by hepatocytes at liver, or g) increasing insulin secretion by pancreatic beta cells and having HPLC Fraction ID Nos. as identified in Table 4 or 8 to 11, and at least one pharmaceutically acceptable carrier, additive, diluent, or excipient.

90. The composition of claim 89, wherein the plant extract has HPLC Fraction ID Nos. as identified in Table 4.

91. The composition of claim 89, wherein the plant extract has HPLC Fraction ID Nos. as identified in Tables 8 to 11.

92. A method of treating a degenerative condition, comprising administering to a subject in need thereof an effective amount of a plant extract having HPLC Fraction ID Nos. as identified in Tables 4 or 8 to 11, thereby (a) lowering glucose level or improving glucose uptake, or increasing insulin sensitivity or (b) increasing respiratory enzyme production and subsequent thermogenesis.

93. The method of claim 92, wherein the degenerative condition is selected from the group consisting of cardiovascular disease, diabetes mellitus, hypertension, cancers, obesity, arthritis, pain, Alzheimer's disease, multiple sclerosis, autoimmune diseases, asthma, allergies, osteoporosis, Parkinson's disease, stress, ocular disorders & diseases of the eye.

94. A plant extract as claimed in claim 86, having insulin mimetic activity and selected from the extracts having HPLC Fraction ID No. selected from:
AVDB008Le04(20)bc6C01A00,
AVDB008Le06bc6C01A00,
AVDB006Se07bc6C01A00 and
AVDB005Le07bc6C01A00

95. A plant extract as claimed in claim 86, having an insulin sensitizing activity and selected from the extracts having HPLC Fraction ID No. selected from:
AVDB006Se04(20)bc6C01A00
AVDB018Fr03bc6C01A00
AVDB003Ro06bc6C01A00

96. A plant extract as claimed in claim 86, having thermogenic activity and HPLC Fraction ID No. AVDB008Le04(20)bc6C01A00.

97. A plant extract as claimed in claim 86, having alpha glucosidase activity and selected from the extracts having HPLC Fraction ID No. selected from:
AVDB006Se08bc6C01A00,
AVDB006Se04(20)bc6C01A00,
AVDB006Se06bc6C01A00 and
AVDB006Se04bc6C01A00

98. The method of claim 92, wherein a plant extract having insulin mimetic activity, alpha glucosidase activity, & insulin sensitizing activity, and having HPLC Fraction ID No. AVDB006Se07bc6C01A00, is administered for the treatment of diabetes.

99. The method of claim 92, wherein a combination of plant extracts having HPLC Fraction ID Nos. AVDB012Ap08bc6C01A00, AVDB021Ba08bc6C01A00 and AVDB006Se04(20)bc6C01A00 is administered.

100. The composition of claim 89, comprising a combination of plant extracts having HPLC Fraction ID Nos. AVDB012Ap08bc6C01A00, AVDB021Ba08bc6C01A00 and AVDB006Se04(20)bc6C01A00.

101. The method of claim 92, wherein a combination of plant extracts having HPLC Fraction ID Nos. AVDB010Fr08bc6C01A00, AVDB021Ba08bc6C01A00, and AVDB006Se04(20)bc6C01A00 is administered.

102. The composition of claim 89, comprising a combination of plant extracts having HPLC Fraction ID Nos. AVDB010Fr08bc6C01A00, AVDB021 Ba08bc6C01A00, and AVDB006Se04(20)bc6C01A00.

103. The method of claim 92, wherein a combination of plant extracts having HPLC Fraction ID Nos. AVDB012Ap08bc6C01A00 and AVDB006Se04(20)bc6C01A00 is administered.

104. The composition of claim 89, comprising a combination of plant extracts having HPLC Fraction ID Nos. AVDB010Fr08bc6C01A00, AVDB021Ba08bc6C01A00, and AVDB006Se04(20)bc6C01A00.

105. The method of claim 92, wherein a combination of plant extracts having HPLC Fraction ID Nos. AVDB025Se08bc6C01A00, AVDB008Lf08bc6C01A00 & AVDB006Se04(20)bc6C01A00 is administered.

106. The composition of claim 89, comprising a combination of plant extracts having HPLC Fraction ID Nos. AVDB025Se08bc6C01A00, AVDB008Lf08bc6C01A00 & AVDB006Se04(20)bc6C01A00.

107. The method of claim 92, wherein a combination of plant extracts having Fraction ID Nos. AVDB006Se04(20)bc6C01A00 & AVDB021Ba08bc6C01A00 is administered.

108. The composition of claim 89, comprising a combination of plant extracts having Fraction ID Nos. AVDB006Se04(20)bc6C01A00 & AVDB021Ba08bc6C01A00.

109. The method of claim 92, wherein a plant extract having HPLC Fraction ID No AVDB006Se08bc6C01A00 is administered.

110. The composition of claim 89, comprising a plant extract having HPLC Fraction ID No AVDB06Se08bc6C01A00.

111. The method of claim 92, wherein a plant extract having HPLC Fraction ID No AVDB006Se04(20)bc6C01A00 is administered.

112. The composition of claim 89, comprising a plant extract having HPLC Fraction ID No AVDB006Se04(20)bc6C01A00.

113. The method of claim 92, wherein a plant extract having HPLC Fraction ID No AVDB019Se08bc6C01A00 is administered.

114. The composition of claim 89, comprising a plant extract having HPLC Fraction ID No AVDB019Se08bc6C01A00.

115. The method of claim 92, wherein a plant extract having HPLC Fraction ID No AVDB019Se04(20)bc6C01A00 is administered.

116. The composition of claim 89, comprising a plant extract having HPLC Fraction ID No AVDB019Se04(20)bc6C01A00.

117. The method of claim 92, wherein a plant extract having HPLC Fraction ID No AVDB008Le08bc6C01A00 is administered.

118. The composition of claim 89, comprising a plant extract having HPLC Fraction ID No AVDB008Le08bc6C01A00.

119. The method of claim 92, wherein a plant extract having HPLC Fraction ID No AVDB008Le04(20)bc6C01A00 is administered.

120. The composition of claim 89, comprising a plant extract having HPLC Fraction ID No AVDB008Le04(20)bc6C01A00.

121. The method of claim 92, wherein a plant extract having HPLC Fraction ID No AVDB013Ba08bc6C01A00 is administered.

122. The composition of claim 89, comprising a plant extract having HPLC Fraction ID No AVDB013Ba08bc6C01A00.

123. The method of claim 92, wherein a plant extract having HPLC Fraction ID No AVDB013Ba04(20)bc6C01A00 is administered.

124. The composition of claim 89, comprising a plant extract having HPLC Fraction ID No AVDB013Ba04(20)bc6C01A00.

125. The method of claim 92, wherein a plant extract having HPLC Fraction ID No AVDB002Ba08bc6C01A00 is administered.

126. The composition of claim 89, comprising a plant extract having HPLC Fraction ID No AVDB02Ba08bc6C01A00.

127. The method of claim 92, wherein a plant extract having HPLC Fraction ID No AVDB005Fr06bc6C01A00 is administered.

128. The composition of claim 89, comprising a plant extract having HPLC Fraction ID No AVDB05Fr06bc6C01A00.

129. The method of claim 92, wherein a plant extract having HPLC Fraction ID No AVDB003Ro06bc6C01A00 is administered.

130. The composition of claim 89, comprising a plant extract having HPLC Fraction ID No AVDB03Ro06bc6C01A00.

131. The method of claim 92, wherein a plant extract having HPLC Fraction ID No AVDB020Ro06bc6C01A00 is administered.

132. The composition of claim 89, comprising a plant extract having HPLC Fraction ID No AVDB020Ro06bc6C01A00.

133. The method of claim 92, wherein a plant extract having HPLC Fraction ID No AVDB019Se06bc6C01A00 is administered.

134. The composition of claim 89, comprising a plant extract having HPLC Fraction ID No AVDB019Se06bc6C01A00.

135. The method of claim 92, wherein a plant extract having HPLC Fraction ID No AVDB006Se06bc6C01A00 is administered.

136. The composition of claim 89, comprising a plant extract having HPLC Fraction ID No AVDB06Se06bc6C01A00.

137. The method of claim 92, wherein a plant extract having HPLC Fraction ID No AVDB002Le06bc6C01A00 is administered.

138. The composition of claim 89, comprising a plant extract having HPLC Fraction ID No AVDB02Le06bc6C01A00.

139. The method of claim 92, wherein a plant extract having HPLC Fraction ID No AVDB018Fr06bc6C01A00 is administered.

140. The composition of claim 89, comprising a plant extract having HPLC Fraction ID No AVDB018Fr06bc6C01A00.

141. The method of claim 92, wherein a plant extract having HPLC Fraction ID No AVDB017Fr06bc6C01A00 is administered.

142. The composition of claim 89, comprising a plant extract having HPLC Fraction ID No AVDB017Fr06bc6C01A00.

143. The method of claim 92, wherein a plant extract having HPLC Fraction ID No AVDB003Ro03bc6C01A00 is administered.

144. The composition of claim 89, comprising a plant extract having HPLC Fraction ID No AVDB03Ro03bc6C01A00.

145. The method of claim 92, wherein a plant extract having HPLC Fraction ID No AVDB020Ro04bc6C01A00 is administered.

146. The composition of claim 89, comprising a plant extract having HPLC Fraction ID No AVDB020Ro04bc6C01A00.

147. The method of claim 92, wherein a plant extract having HPLC Fraction ID No AVDB020Ro03bc6C01A00 is administered.

148. The composition of claim 89, comprising a plant extract having HPLC Fraction ID No AVDB020Ro03bc6C01A00.

149. The method of claim 92, wherein a plant extract having HPLC Fraction ID No AVDB019Seo04bc6C01A00 is administered.

150. The composition of claim 89, comprising a plant extract having HPLC Fraction ID No AVDB019Seo04bc6C01A00.

151. The method of claim 92, wherein a plant extract having HPLC Fraction ID No AVDB04Se04bc6C01A00 is administered.

152. The composition of claim 89, comprising a plant extract having HPLC Fraction ID No AVDB04Se04bc6C01A00.

153. The method of claim 92, wherein a plant extract having HPLC Fraction ID No AVDB018Fr04bc6C01A00 is administered.

154. The composition of claim 89, comprising a plant extract having HPLC Fraction ID No AVDB018Fr04bc6C01A00.

155. The method of claim 92, wherein a plant extract having HPLC Fraction ID No AVDB011Fr04bc6C01A00 is administered.

156. The composition of claim 89, comprising a plant extract having HPLC Fraction ID No AVDB011Fr04bc6C01A00.

157. The method of claim 92, wherein a plant extract having HPLC Fraction ID No AVDB017Fr04bc6C01A00 is administered.

158. The composition of claim 89, further comprising a plant extract having HPLC Fraction ID No AVDB017Fr04bc6C01A00.

159. The method of claim 92, wherein a plant extract having HPLC Fraction ID No AVDB019Se03bc6C01A00 is administered.

160. The composition of claim 89, comprising a plant extract having HPLC Fraction ID No AVDB019Se03bc6C01A00.

161. The method of claim 92, wherein a plant extract having HPLC Fraction ID No AVDB006Se03bc6C01A00 is administered.

162. The composition of claim 89, comprising a plant extract having HPLC Fraction ID No AVDB06Se03bc6C01A00.

163. The method of claim 92, wherein a plant extract having HPLC Fraction ID No AVDB011Fr03bc6C01A00 is administered.

164. The composition of claim 89, comprising a plant extract having HPLC Fraction ID No AVDB011Fr03bc6C01A00.

165. The method of claim 92, wherein a plant extract having HPLC Fraction ID No AVDB017Fr03bc6C01A00 is administered.

166. The composition of claim 89, comprising a plant extract having HPLC Fraction ID No AVDB017Fr03bc6C01A00.

167. The method of claim 92, wherein a plant extract having HPLC Fraction ID No AVDB005Fr03bc6C01A00 is administered.

168. The composition of claim 89, comprising a plant extract having HPLC Fraction ID No AVDB005Fr03bc6C01A00.

169. The method of claim 92, wherein a plant extract having HPLC Fraction ID No AVDB008Le06bc6C01A00 is administered.

170. The composition of claim 89, comprising a plant extract having HPLC Fraction ID No AVDB08Le06bc6C01A00.

171. The method of claim 92, wherein a plant extract having HPLC Fraction ID No AVDB006Fr03bc6C01A00 is administered.

172. The composition of claim 89, comprising a plant extract having HPLC Fraction ID No AVDB06Fr03bc6C01A00.

173. The method of claim 92, wherein a plant extract having HPLC Fraction ID No AVDB020Ro07bc6C01A00 is administered.

174. The composition of claim 89, comprising a plant extract having HPLC Fraction ID No AVDB020Ro07bc6C01A00.

175. The method of claim 92, wherein a plant extract having HPLC Fraction ID No AVDB019Se07bc6C01A00 is administered.

176. The composition of claim 89, comprising a plant extract having HPLC Fraction ID No AVDB019Se07bc6C01A00.

177. The method of claim 92, wherein a plant extract having HPLC Fraction ID No AVDB006Se07bc6C01A00 is administered.

178. The composition of claim 89, comprising a plant extract having HPLC Fraction ID No AVDB06Se07bc6C01A00.

179. The method of claim 92, wherein a plant extract having HPLC Fraction ID No AVDB018Fr07bc6C01A00 is administered.

180. The composition of claim 89, comprising a plant extract having HPLC Fraction ID No AVDB018Fr07bc6C01A00.

181. The method of claim 92, wherein a plant extract having HPLC Fraction ID No AVDB008Le07bc6C01A00 is administered.

182. The composition of claim 89, comprising a plant extract having HPLC Fraction ID No AVDB08Le07bc6C01A00.

183. The method of claim 92, wherein a plant extract having HPLC Fraction ID No AVDB002Le05bc6C01A00 is admininstered.

184. The composition of claim 89, comprising a plant extract having HPLC Fraction ID No AVDB02Le05bc6C01A00.

185. The method of claim 92, wherein a plant extract having HPLC Fraction ID No AVDB020Ro02bc6C01A00 is administered.

186. The composition of claim 89, comprising a plant extract having HPLC Fraction ID No AVDB020Ro02bc6C01A00.

187. The method of claim 92, wherein a plant extract having HPLC Fraction ID No AVDB019Se02bc6C01A00 is administered.

188. The composition of claim 89, comprising a plant extract having HPLC Fraction ID No AVDB019Se02bc6C01A00.

189. The method of claim 92, wherein a plant extract having HPLC Fraction ID No AVDB002Se02bc6C01A00 is administered.

190. The composition of claim 89, comprising a plant extract having HPLC Fraction ID No AVDB02Se02bc6C01A00.

191. The method of claim 92, wherein a plant extract having HPLC Fraction ID No AVDB018Fr02bc6C01A00 is administered.

192. The composition of claim 89, comprising a plant extract having HPLC Fraction ID No AVDB018Fr02bc6C01A00.

193. The method of claim 92, wherein a plant extract having HPLC Fraction ID No AVDB008Le06bc6C01A00 is administered.

194. The composition of claim 89, comprising a plant extract having HPLC Fraction ID No AVDB08Le06bc6C01A00.

195. The method of claim 92, wherein a plant extract having HPLC Fraction ID No AVDB002Le02bc6C01A00 is administered.

196. The composition of claim 89, comprising a plant extract having HPLC Fraction ID No AVDB02Le02bc6C01A00.

197. The method of claim 92, wherein a plant extract having HPLC Fraction ID No AVDB020Le08bc6C01A00 is administered.

198. The composition of claim 89, comprising a plant extract having HPLC Fraction ID No AVDB020Le08bc6C01A00.

199. The method of claim 92, wherein a plant extract having HPLC Fraction ID No AVDB008Le04bc6C01A00 is administered.

200. The composition of claim 89, comprising a plant extract having HPLC Fraction ID No AVDB08Le04bc6C01A00.

201. The method of claim 92, wherein a plant extract having HPLC Fraction ID No AVDB002Le03bc6C01A00 is administered.

202. The composition of claim 89, comprising a plant extract having HPLC Fraction ID No AVDB02Le03bc6C01A00.

203. The method of claim 92, wherein a plant extract having HPLC Fraction ID No AVDB002Le04bc6C01A00 is administered.

204. The composition of claim 89, comprising a plant extract having HPLC Fraction ID No AVDB002Le04bc6C01A00.

205. The method of claim 92, wherein a plant extract having HPLC Fraction ID No AVDB002Le07bc6C01A00 is administered.

206. The composition of claim 89, comprising a plant extract having HPLC Fraction ID No AVDB02Le07bc6C01A00.

207. The method of claim 92, wherein a plant extract having HPLC Fraction ID No AVDB018Fr03bc6C01A00 is administered.

208. The composition of claim 89, comprising a plant extract having HPLC Fraction ID No AVDB018Fr03bc6C01A00.

209. The method of claim 92, wherein a plant extract having HPLC Fraction ID No AVDB008Le02bc6C01A00 is administered.

210. The composition of claim 89, comprising a plant extract having HPLC Fraction ID No AVDB08Le02bc6C01A00.

211. The method of claim 92, wherein a plant extract having HPLC Fraction ID No AVDB003Ro04bc6C01A00 is administered.

212. The composition of claim 89, comprising a plant extract having HPLC Fraction ID No AVDB03Ro04bc6C01A00.

213. The method of claim 92, wherein a plant extract having HPLC Fraction ID No AVDB008Le03bc6C01A00 is administered.

214. The composition of claim 89, comprising a plant extract having HPLC Fraction ID No AVDB08Le03bc6C01A00.

215. The method of claim 92, wherein a plant extract having HPLC Fraction ID No AVDB013Ba04bc6C01A00 is administered.

216. The composition of claim 89, comprising a plant extract having HPLC Fraction ID No AVDB013Ba04bc6C01A00.

217. The method of claim 92, wherein a plant extract having HPLC Fraction ID No AVDB008Le09bc6C01A00 is administered.

218. The composition of claim 89, comprising a plant extract having HPLC Fraction ID No AVDB08Le09bc6C01A00.

219. The method of claim 92, wherein a plant extract having HPLC Fraction ID No AVDB008Le01bc6C01A00 is administered.

220. The composition of claim 89, comprising a plant extract having HPLC Fraction ID No AVDB008Le01bc6C01A00.

221. The method of claim 92, wherein a plant extract having HPLC Fraction ID No AVDB019Se09bc6C01A00 is administered.

222. The composition of claim 89, comprising a plant extract having HPLC Fraction ID No AVDB019Se09bc6C01A00.

223. The method of claim 92, wherein a plant extract having HPLC Fraction ID No AVDB019Se01bc6C01A00 is administered.

224. The composition of claim 89, comprising a plant extract having HPLC Fraction ID No AVDB019Se01bc6C01A00.

225. The method of claim 92, wherein a plant extract having HPLC Fraction ID No AVDB005Le07bc6C01A00 is administered.

226. The composition of claim 89, comprising a plant extract having HPLC Fraction ID No AVDB05Le07bc6C01A00.

227. The method of claim 92, wherein a plant extract having HPLC Fraction ID No AVDB005Le05bc6C01A00 is administered.

228. The composition of claim 89, comprising a plant extract having HPLC Fraction ID No AVDB005Le05bc6C01A00.

229. The method of claim 92, wherein a plant extract having HPLC Fraction ID No AVDB006Se05bc6C01A00 is administered.

230. The composition of claim 89, comprising a plant extract having HPLC Fraction ID No AVDB06Se05bc6C01A00.

231. A successive plant extract demonstrating at least one of a) insulin mimetic activity, b) insulin sensitizing activity, c) alpha glucosidase activity, d) thermogenesis activity, e) enhanced glucose uptake activity by fat cells or peripheral skeletal muscle cells, f) decreasing glucose secretion by hepatocytes at liver and g) increasing insulin secretion by pancreatic beta cells, having an HPLC Fraction ID Nos. as identified in Table 12.

232. A composition comprising an antioxidant effective amount of at least one successive plant extract demonstrating at least one of a) insulin mimetic activity, b) insulin sensitizing activity, c) alpha glucosidase activity, d) thermogenesis activity, e) enhanced glucose uptake activity by fat cells or peripheral skeletal muscle cells, f) decreasing glucose secretion by hepatocytes at liver and g) increasing insulin secretion by pancreatic beta cells and having HPLC Fraction ID Nos. as identified in Table 12, and at least one pharmaceutically acceptable carrier, additive, diluent, or excipient.

233. A composition comprising an antioxidant effective amount of at least one derivative molecule of a bioactive obtained from the successive plant extract as shown in Table 12, the molecule demonstrating at least one of a) insulin mimetic activity, b) insulin sensitizing activity, c) alpha glucosidase activity, d) thermogenesis activity, e) enhanced glucose uptake activity by fat cells or peripheral skeletal muscle cells, f) decreasing glucose secretion by hepatocytes at liver and g) increasing insulin secretion by pancreatic beta cells, and at least one pharmaceutically acceptable carrier, additive, diluent, or excipient.
